(19) United States
(12) Patent Application Publication
(10) Pub. No.: US 2006/0260557 A1
(43) Pub. Date: Nov. 23, 2006

(54) Systems and methods for improving efficiencies in avian species

(76) Inventors: Michael McCabe, Dacula, GA (US); Thomas Mathieu Zwercher, Lyon (FR); Jean Beausire, St. Sorlin (FR); Stephane Lemiere, Lyon (FR)

Correspondence Address: Judy Jarecki-Black, Ph.D, J.D, 3239 Satellite Blvd., Duluth, GA, 30096, US

(21) Appl. No.: 11/269,782

(22) Filed: Nov. 8, 2005

Related U.S. Application Data
(60) Provisional application No. 60/625,854, filed on Nov. 8, 2004.

(51) Int. Cl. A01K 29/00

(52) U.S. Cl. 119/174

(57) ABSTRACT

The present invention provides computer-assisted methods, including interactive systems, for tracking and correlating of breeding, veterinary care and performance data relating to livestock animals. In particular, the methods are especially useful for tracking breeding and veterinary care histories of individual animals or the collective herd or flock and relating the histories to performance parameters of the animals and to expected or desired performances. The methods of the invention can allow the operator to be alerted by deviation of the actual performance parameters from desired performances so that feed, medications, vaccinations, husbandry conditions, food safety etc of the animals may be adjusted or corrected accordingly. Such methods include obtaining and maintaining the data obtained from each animal, and optionally other data relating to the animal's health, condition or parentage, or to its herd, and providing this data to others through web-based systems, contained in a database, or attached to the animal itself such as by an implanted microchip.

This application claims priority from U.S. provisional application 60/625,854 filed Nov. 8, 2004, which is incorporated herein by reference in its entirety

### INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to methods and systems of identification and management of avian flocks. More specifically, the invention relates to methods and systems including network-based processes to manage data such as identification and traceability of data relating to specific avian flocks, veterinarian care, diagnostic and quality control data and management of flocks, husbandry conditions, animal welfare, food safety information, audit of existing processes and data from field locations. The invention further relates to interactive systems suitable for inputting responses from system operators.

### BACKGROUND OF THE INVENTION

The body condition of animals is a determinant of market readiness in commercial livestock breeding, feeding and finishing operations. “Body condition” as understood in the livestock industry is the state of development of an animal as a function of frame type or size, and overall health. In the case of non-poultry animals, the amount of intramuscular fat and back fat exhibited by an animal. It is typically determined subjectively and through experienced visual appraisal of live animals. Weight and visual clues are one means possible to sort cattle for feed lot grouping. Poultry may be characterized by, for example, by muscular mass, rate of growth, egg laying potential, etc.

Regardless of the particular market preference at a given time, the producer will be trying to tailor animals to meet consistent standards that will cause a meat packer or commercial purchaser to pay the highest price in accordance with currently prevailing market preferences. Animals used for dairy milk production or birds for egg production are priced according to yield expectations. The greater the production expectations, the greater the price realized by the feed operator.

While the cost of acquiring each animal in a group can vary, feeding costs are the same for each animal in a group since they can be fed the same amount of feed and occupy space in the house or shed for the same amount of time. Thus, the price reductions for animals falling outside the desirable range fall directly to the producer's bottom line, resulting in reduced profits. One way for the breeder to reduce costs and increase profits is to alter the time an animal spends on the lot, thus reducing the feed costs. Longer residence times are usually only profitable if the result is an animal with a more profitable grade. The capability of predicting when a bird or an animal is ready for a market is also desirable.

An important consideration for animal management practices is the treatment and prevention of infectious diseases. Current production practice involves prophylactic treating of animals en masse for infectious diseases, or when clinical signs arise, because individual animals at risk cannot be easily identified.

It is desirable to track veterinary care, husbandry and health conditions of livestock animals to optimize the growth and yield of the animals, and to compare the health and performance parameters with the desired goals of the industry. Also desirable is a method that allows for the compilation, retrieval, and sharing of veterinary information in a database such that the data pertaining to disease, health conditions, phenotypic traits of economic importance, husbandry conditions, the vaccination and medication records and the like, is available upon accessing the database. Such data could be available from a central database or coded, for example, on a chip implanted in the individual animal and tracked according to the flock, herd or farm associated with the animal.

Because of these deficiencies and others inherent in the prior art, it would be advantageous to provide a business method that provides for increased production efficiencies in poultry and livestock animals such as cattle, swine, sheep and the like, as well as providing access to various records of the animals and allows comparisons with expected or desired goals with regard to the quality and quantity of animals produced. It would be advantageous to provide interactive systems whereby, during the training of an operator of the business method, the instructor can obtain immediate responses to questions during the training period and thereby adapt the course material to pupil's progress. It would also be useful for an interactive system to allow a salesperson to introduce a potential commercial product to the user of the business method and monitor the response of the potential customer audience before and after the effect of a product upon livestock yield is apparent. An interactive computer-based business method would be particularly advantageous for an operator in the field to access databases pertaining to a subject flock of birds or group of animals, modify the data in the database or receive information concerning such as appropriate medication to be administered to the animals.

### SUMMARY OF THE INVENTION

Citation or identification of any document in this application is not an admission that such document is available as prior art to the present invention.

The present invention is directed to computer-assisted methods and systems for improving the efficiency of livestock production, advantageously of poultry, using multiple data obtained from the animals, the husbandry conditions and the expected goals. Methods of the invention include obtaining and maintaining data relating to the animal's husbandry conditions, health and veterinary care, condition or parentage, or to its flock, and providing this data to others through systems that are web-based, contained in a database, or attached to the animal itself such as by an implanted microchip. Methods of the invention may also comprise obtaining a genetic sample from each animal in a flock, determining the genotype of each bird with respect to specific quality traits, grouping birds with like genotypes, and optionally, further sub-grouping animals based on like phenotypes.

The present invention encompasses computer-assisted methods for collecting and storing data including, for example, husbandry conditions, breeding, vaccination, medication, and feed data, genotyping or classifying livestock based on the genetic data, and formulating feed, medication and slaughter schedules for the birds. The methods of the present invention may optimize the efficiencies of raising poultry since the producer or packer can predict optimum husbandry conditions, vaccination, medication and feed quantities and, if and when required, slaughter schedules for each animal or group of animals. Conditions may also be optimized for the laying of eggs by poultry. The computer-assisted methods may further encompass interactive systems that may allow an operator such as a student, customer or filed operator to input responses to questions posed by a third-party during the course of training or operation of the system in the field, and so permitting immediate modification of a training program or a parameter that directly affects the raising of the livestock or birds. For example, veterinarians accessing the system by means of a potable computing device such as a PDA or internet-connected pocket computer may modify a medication regimen to the specific needs of the subject animals.

The present invention also encompasses computer-assisted methods and systems for acquiring genetic data, particularly data related to health and quality traits of the breed of animal and associating that data with other data about the animal or its herd, and maintaining that data in ways that are accessible.

One aspect of the present invention, therefore, encompasses computer-assisted methods for the tracking and correlating of husbandry conditions, breeding, veterinary care and performance data relating to one or more animals. The methods are especially useful for tracking the breeding and veterinary care histories of individual animals or the collective flock and relating the histories to the performance parameters of the animals and to the expected or desired performances. The methods of the invention can allow the operator to be alerted by deviation of the actual performance parameters from the desired performances so that the feed, medications, vaccinations etc of the animals may be adjusted or corrected accordingly. The methods of the invention allow the operator to interact with central servers and databases, thereby accessing data relevant to a particular flock or group of animals.

One embodiment of the invention encompasses an interactive computer-assisted method for tracking the breeding, husbandry conditions and veterinary histories of poultry comprising using a computer system, such as a programmed computer encompassing a processor, a data storage system, an input device and an output device, and comprising the steps of inputting into the programmed computer through the input device data comprising a breeding history of a bird or flock of birds, inputting into the programmed computer through the input device data comprising a veterinary history of a bird or flock of birds, correlating the veterinary data with the breeding history of the bird or flock of birds using the processor and the data storage system and outputting to the output device the breeding history and the veterinary history of the bird or flock of birds.

In one embodiment of the invention, the method further comprises the steps of inputting into the programmed computer diagnostic data related to the health of the livestock animal or population of bird or flock of birds; and correlating the diagnostic data to the breeding and veterinary histories of the livestock animal or population of bird or flock of birds.

In the various embodiments of the invention, the veterinary data can include, but is not limited to, a vaccination record for a bird or flock of birds. The health data may comprise other than the vaccination record for a livestock animal or population of poultry such as, but not limited to, records of medications administered to the animal(s), doses, infections or other injuries and surgical procedures.

In other embodiments of the methods according to the invention, steps may be included of inputting into the programmed computer data related to the welfare and husbandry conditions of the bird or flock of birds; and correlating the welfare data to the breeding and veterinary histories of the bird or flock of birds.

In still other embodiments of the invention, the methods may further comprise the steps of inputting into the programmed computer data related to the quality control of the bird or flock of birds, including food safety, and correlating this quality control data to the breeding, husbandry conditions and veterinary histories of the bird or flock of birds.

The method according to the invention may also comprise the steps of inputting into the programmed computer performance parameters of the bird or flock of birds and correlating the required performance parameters of the bird or flock of birds to a specific desired performance requirement of a customer. The embodiments may optionally further comprise correlating vaccine data to the performance parameters of the bird or flock of birds and/or inputting into the programmed computer data related to the nutritional data of the bird or flock of birds, husbandry conditions, and correlating the nutritional data to the performance parameters of the bird or flock of birds.

Yet other embodiments of method according to the invention further comprise the step of alerting the system operator to undesirable changes in the performance parameters of the bird or flock of birds and which would allow the operator to modify the feed, husbandry conditions, or medication of the animals accordingly. The embodiments of the invention may further comprise inputting into the programmed computer through the input device data comprising a genotype of an animal, correlating a physical characteristic predicted by the genotype using the processor and the data storage system, and outputting to the output device the physical characteristic correlated to the genotype for a bird or flock of birds and feeding the animal(s) a diet based upon the physical characteristic, thereby improving livestock production.

Still other embodiments of the computer-assisted methods according to the invention optimize the efficiency of feed lots for livestock and comprise outputting to an output device the breeding and veterinary history of the bird or flock of birds, and feeding the animal(s) a diet based upon their breeding and veterinary histories, thereby optimizing efficiency of feed lots for the bird or flock of birds.

It is contemplated to be within the scope of the invention for the computer-assisted methods of the present invention to be suitable for collating, analyzing and providing data on any livestock animal including, but not limited to a bird or flock of birds such as chicken, turkey, duck and the like as well as sheep, cattle, swine and the like.

Another aspect of the invention is a computer system for tracking breeding and welfare histories of livestock comprising breeding and veterinarian data corresponding to one or more livestock animals. Yet another aspect of the invention is computer readable media for tracking breeding and welfare histories of livestock comprising breeding and veterinarian data corresponding to one or more livestock animals. Still another aspect of the invention is methods of doing business for tracking breeding and welfare histories of livestock comprising breeding and veterinarian data corresponding to one or more livestock animals comprising providing to a user the computer system of claim 14 or the media of claim 15.

Also provided by the present invention is a method that allows for the compilation, retrieval, and sharing of genetic information in a database such that genetic predispositon to disease, health conditions, and other phenotypic traits of economic importance is available upon accessing the database. Such data could be available from a central database or coded, for example, on a chip implanted in the individual animal.

Another aspect of the present invention is a method of transmitting data comprising transmission of information from such methods herein discussed or steps thereof, e.g., via telecommunication, telephone, video conference, mass communication, e.g., presentation such as a computer presentation such as a POWERPOINT™ presentation, internet, email, documentary communication such as a computer program (e.g. WORD) document and the like. It is further contemplated that this aspect of the invention may encompass an interactive system for the exchange or information such as student responses to questions posed by an instructor that would allow the instructor to promptly modify a presentation or the output of information from the computer-assisted methods, thereby adapting the computer system of the invention to the specific needs of the operator.

Another aspect of the invention is a method for entering and retrieving data relevant to a flock of birds into a computer device such as, but not limited to, a personal digital assistant (PDA) or a pocket computer that may be readily transported to the location of the flock to provide instant access by the operator to information relevant to the maintenance and rearing of the birds. The method may also provide a firewall protected access to the internet to further expand the available information and to allow suppliers of, for example, veterinarian products to suggest suitable products and dosages useful for administering to the birds. The computer device may also be configured to synchronize with a central server to deliver data to the device and to permit the uploading of data to the server for storage and/or subsequent analysis. The methods of the invention as incorporated into a readily portable computer device are especially useful to a veterinarian making field visits to the site of the bird rearing house or shed. The software of the method may be configured to allow veterinarians to set their personal data and preferences, including name, address, telephone number and the like.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates a flowchart of the input of data and the output of results from the analysis and correlation of the data pertaining to the breeding, veterinarian histories and performance requirements of a group of animals such as from a flock of birds and the interactive flow of data from the computer-assisted device to a body of students learning the use of the method of the invention.

FIG. 2 illustrates a flowchart for the input of data detailing the breeding history and the vaccination records of a flock of birds.

FIG. 3 illustrates potential relationships between the data elements to be entered into the system. Unidirectional arrows indicate, for example, that a house or shed is typically owned by only one farm, whereas a farm may own several houses or sheds. Similarly, a prescription may include have several veterinarian products.

FIG. 4A illustrates the flow of events in the use of the portable computer-based system for data entry on the breeding and rearing of a flock of birds.

FIG. 4B illustrates the flow of events through the sub-routines related to data entry concerning farm management.

FIG. 4C illustrates the flow of events through the sub-routines related to data entry concerning data specific to a company.

FIG. 5 illustrates a flow chart of the input of data and the output of results from the analysis and the correlation of the data pertaining to the breeding, veterinarian histories, and performance requirements of a group of animals.

### DETAILED DESCRIPTION OF THE INVENTION

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

In the description that follows, a number of terms are extensively utilized. In order to provide a clear and consistent understanding of the specification and claims, including the scope to be given such terms, the following terminology is provided:

The term “animal” is used herein to include all vertebrate animals, including humans. It also includes an individual animal in all stages of development, including embryonic and fetal stages. As used herein, the term “production animals” is used interchangeably with “livestock animals” and refers generally to animals raised primarily for food, either as meat, meat products or as eggs or egg products. The animals as referred to herein may also include individuals or groups of individuals that are raised for other than food production such as, but not limited to, transgenic animals for the production of biopharmaceuticals including antibodies and other proteins or protein products. For example, such animals include, but are not limited to, cattle (bovine), sheep (ovine), pigs (porcine or swine), poultry (avian), and the like. As used herein, the term “cow” or “cattle” is used generally to refer to an animal of bovine origin of any age. Interchangeable terms include “bovine”, “calf”, “steer”, “bull”, “heifer” and the like. As used herein, the term “pig” or “swine” is used generally to refer to an animal of porcine origin of any age. Interchangeable terms include “piglet”, “sow” and the like. As used herein, the terms “avian” and “poultry” refer to any bird such as, but not limited to, chicken, turkey, duck, goose, pheasant, grouse, ostrich and quail raised for food, egg production, therapeutic protein production and the like.

As used herein, the terms “quality traits” or “physical characteristics” or “phenotypes” refer to advantageous properties of the animal resulting from genetics. Quality traits include, but are not limited to, the animal's genetic ability to efficiently metabolize energy, produce meat or milk (non-avian species), put on intramuscular fat, lay eggs (avians), produce offspring, produce particular proteins in meat or milk, retain protein in milk, resist disease or produce an exogenous protein not typically found in the animal or egg thereof. The terms are used interchangeably.

The term “breeding history” as used herein refers to a record of the life of an animal or group of animals including, but not limited to the location, breed, period of housing, individual number allocated to a particular bird, rate of egg laying, fertility, hatchability, mortality and the like, as well as a genetic history of the animals, including parentage and descent therefrom, genotype, phenotype, transgenic history if relevant and the like.

The term “husbandry conditions” as used herein refers to parameters relating to the maintenance of animals including, but not limited to, shed or housing temperature, weekly mortality of a herd or flock, water consumption, feed consumption, ventilation rate and quality, litter condition and the like.

The term “veterinary history” as used herein refers to vaccination data of an animal or group of animals, including, but not limited to, vaccine type(s), vaccine batch serial number(s), administered dose, target antigen, method of administering of the vaccine to the recipient animal(s), number of vaccinated animals, age of the animals and the vaccinator. Data relating to a serological or immunological response induced by the vaccine may also be included. “Veterinary history” as used herein is also intended to include the medication histories of the target animal(s) including, but not limited to drug and/or antibiotics administered to the animals including type of administered medication, quantity and dose rates, by whom and when administered, by what route, e.g., oral, subcutaneously and the like, and the response to the medication. Husbandry conditions and veterinary conditions for each stage of bird rearing may be combined. For example (as illustrative examples only and not intended to be limiting), for the pullet farm: location, breed, day of placement, number birds, uniformity, livability, body weight, vaccination (vaccine, serial number, dose, antigen, method, number of birds, date, age, vaccinator), medication, audit may be included; for the breeder farm: location, breed, day of placement, number birds, rate of lay, livability, body weight, vaccination, vaccine, serial number, dose, antigen, method, number of birds, date, age, vaccinator), medication, audit; the hatchery: date of hatch, breeder farm, fertility, hatchability, vaccination (vaccine, serial number, dose, antigen, method, number of birds, date, age, vaccinator), audit may be included; for the broiler farm, day of placement, breeder farm, number birds, hatchery, early mortality (3-7 days), vaccination (vaccine, serial number, dose, antigen, method, date, age, vaccinator), medication, audit, husbandry conditions (house temperature, weekly mortality, water consumption, feed consumption, ventilation, air quality, litter condition, bird weights, cocci checks) may be included; and for diagnostic lab: breeder serology, broiler processing, other serology, other parameters may be included.

The term “diagnostic data” as used herein refers to data relating to the health of the animal(s) other than data detailing the vaccination or medication history of the animal(s). For example, the diagnostic data may be a record of the infections experienced by the animal(s) and the response thereof to medications provided to treat such medications. Serological data including antibody or protein composition of the serum or other biofluids may also be diagnostic data useful to input in the methods of the invention. Surgical data pertaining to the animal(s) may be included, such as the type of surgical manipulation, outcome of the surgery and complications arising from the surgical procedure. “Diagnostic data” may also include measurements of such parameters as weight, morbidity, and other characteristics noted by a veterinary service such as the condition of the skin, feet, feather density, egg laying etc.

The term “welfare data” as used herein refers to parameters such as weight, meat density, crowding levels in breeding or rearing enclosures, psychological behavior of the animal, growth rate, egg laying rate and quality and the like.

The term “quality control” as used herein refers to the desired characteristics of the animal(s). For example, for poultry this can mean, but is not limited to, muscle density, fat content and quantity, egg laying capacity, transgenic protein product yield and the like. For non-poultry animals such as cattle and sheep for example, such parameters include muscle quantity and density, fat content, and the like.

The term “performance parameters” as used herein refers to such factors as meat yield, breeding yield and the like that may be the desired goals from the breeding and rearing of the animal(s). Performance parameters may be either generated from the animals themselves, or those parameters desired by a customer or the market.

The term “nutritional data” as used herein refers to the composition, quantity and frequency of delivery of feed, including water, provided to the animal(s).

The term “food safety” as used herein refers to the quality and quantity of the meat from a bird or livestock animal, including, but not limited to, preparation time, place and manner, storage of the food product, transportation route, inspection records, texture, color, taste, odor, bacterial content, parasitic content and the like.

The term “interactive system” as used herein refers to a computer-based system comprising a means of inputting data into the system. The data input may be in response to a demand such as a question posed to the operator. The interactive system, therefore, may encompass an output in the form of answers to questions relating to the ability of a student to understand or operate the computer system. The interactive system of the invention may encompass a means of receiving the input from multiple stations such as from multiple students, the means of inputting being, for example a wireless transmitter configured to allow a student to respond to a multiple-choice question presented by the instructor. The input of the responses from the students may be collated and presented, for example, as a numeric value or graphically, such as but not limited to, a pie-chart, bar chart and the like. The interactive system may allow an operator such as an instructor to promptly modify the instruction of the computer-based system according to the progress or expectations of the students. It may also allow an operator such a veterinarian in the field to access or enter data concerning a subject flock or group of animals into a database, interact with a system manager and modify treatment protocols etc as indicated or advised according to the specific needs of a subject flock. It is anticipated that one suitable computer-based system for the exchange of information using the methods of the invention may be a personal digital assistant (PDA), a pocket computer or the like that may be connected to a central server by a wireless system. The interactive system as understood herein may also be any device known to one of skill in the art that will allow for the input of simple data into the computer-assisted method of the invention, including, but not limited to, hand-held wireless or cable-connected devices comprising a button or switch means to set a desired response and the necessary electronics and software for transmitting the response in an electronic or numeric form.

A “computer system” refers to the hardware means, software means and data storage means used to compile the data of the present invention. The minimum hardware means of computer-based systems of the invention may comprise a central processing unit (CPU), input means, output means, and data storage means. Desirably, a monitor is provided to visualize structure data. The data storage means may be RAM or other means for accessing computer readable media of the invention. Examples of such systems are microcomputer workstations available from Silicon Graphics Incorporated and Sun Microsystems running Unix based, Linux, Windows NT, XP or IBM OS/2 operating systems. A computer system or computer-based system as used herein may also refer to a portable device such as a PDA or pocket computer that is readily transportable to a site of animal rearing and connected to a central server either by cable or by a wireless connection.

“Computer readable media” refers to any media which can be read and accessed directly by a computer, and includes, but is not limited to: magnetic storage media such as floppy discs, hard storage medium and magnetic tape; optical storage media such as optical discs or CD-ROM; electrical storage media such as RAM and ROM; and hybrids of these categories, such as magnetic/optical media. By providing such computer readable media, the data compiled on a particular animal can be routinely accessed by a user such as a veterinarian or a feedlot operator.

The term “data analysis module” is defined herein to include any person or machine, individually or working together, which analyzes the sample and determines the genetic information contained therein. The term may include a person or machine within a laboratory setting.

As used herein, the term “data collection module” refers to any person, object or system obtaining a tissue sample from an animal or embryo. By example and without limitation, the term may define, individually or collectively, the person or machine in physical contact with the animal as the sample is taken, the containers holding the tissue samples, the packaging used for transporting the samples, and the like. Advantageously, the data collector is a person. More advantageously, the data collector is a livestock farmer, a breeder or a veterinarian

The term “network interface” is defined herein to include any person or computer system capable of accessing data, depositing data, combining data, analyzing data, searching data, transmitting data or storing data. The term is broadly defined to be a person analyzing the data, the electronic hardware and software systems used in the analysis, the databases storing the data analysis, and any storage media capable of storing the data. Non-limiting examples of network interfaces include people, automated laboratory equipment, computers and computer networks, data storage devices such as, but not limited to, disks, hard drives or memory chips.

The individual data of each bird or flock can be recorded and associated with various other data of the animal, e.g. health information, parentage, husbandry conditions, vaccination history, herd or flock records, subsequent food safety data and the like. Such information can be forwarded to a government agency to provide traceability of an animal or meat product, or it may serve as the basis for breeding, feeding and marketing information. Once the data has or has not been associated with other data, the data may be stored in an accessible database, such as a computer database accessible by an internet or VPN link or a microchip implanted in the animal. The methods of the invention may provide an analysis of the input data that may be compared with parameters desired by the operator, such as breeding goals, egg laying targets, vaccination levels of a flock or herd. If the performance or properties of the animals deviates from the desired goals, the computer-based methods may trigger an alert to allow the operator to adjust vaccination doses, medications, feed etc accordingly.

The results of the analysis provide data that can be associated with the individual bird or flock in whole or in part from which the sample was taken. The data can then kept in an accessible database, and may or may not be associated with other data from that particular individual or from other animals.

Data obtained from individual animals is stored in a database which can be integrated or associated with and/or cross-matched to other databases. The database along with the associated data allows information about the individual animal to be known through every stage of the animal's life, i.e., from conception to consumption of the animal product.

The accumulated data and the combination of the genetic data with other types of data of the animal provides access to information about parentage, identification of herd or flock, health information including vaccinations, exposure to diseases, feed lot location, diet and ownership changes. Information such as dates and results of diagnostic or routine tests are easily stored and attainable. Such information would be especially valuable to companies, particularly those who seek superior breeding lines.

Each animal may be provided with a unique identifier. The animal can be tagged, as in traditional tracing programs or have implant computer chips providing stored and readable data or provided with any other identification method which associates the animal with its unique identifier. For example, an animal may be associated with a radio-frequency identification tag (RFID) that can be detected and transfer data by a radio wave.

The database containing the genotype results for each animal or the data for each animal can be associated or linked to other databases containing data, for example, which may be helpful in selecting traits for grouping or sub-grouping of an animal. For example, and not for limitation, data pertaining to birds grouped for propensity to lay can be linked with data pertaining to animals having particular vaccination or medication protocols, and optionally can be further linked with data pertaining to animals having food from certain food sources. The ability to refine a group of animals is limited only by the traits sought and the databases containing information related to those traits.

Databases that can usefully be associated with the methods of the invention include, but are not limited to, specific or general scientific data. Specific data includes, but is not limited to, breeding lines, sires, dames, and the like, other animals' genotypes, including whether or not other specific animals possess specific genes, including transgenic genetic elements, location of animals which share similar or identical genetic characteristics, and the like. General data includes scientific data such as which genes encode for specific quality characteristics, breed association data, feed data, breeding trends, and the like.

A method of the present invention includes providing the animal owner or customer with sample collection equipment, such as swabs and vials useful for collecting samples from which genetic data may be obtained. The vials are packaged in a container which is encoded with identifying indicia. Advantageously, the packaging is encoded with a bar code label. The vials are encoded with the same identifying indicia, advantageously with a matching bar code label. Optionally, the packaging contains means for sending the vials to a laboratory for analysis. The optional packaging is also encoded with identifying indicia, advantageously with a bar code label.

The method optionally includes a system wherein a database account is established upon ordering the sampling equipment. The database account identifier corresponds to the identifying indicia of the vials and the packaging. Upon shipment of the sampling equipment in fulfillment of the order, the identifying indicia are recorded in a database. Advantageously, the identifier is a bar code label which is scanned when the vials are sent. When the vials are returned to the testing facility, the identifier is again recorded and matched to the information previously recorded in the database upon shipment of the vial to the customer. Once the genotyping is completed, the information is recorded in the database and coded with the unique identifier. Test results are also provided to the customer or animal owner.

The data stored in the genotype database can be integrated with or compared to other data or databases for the purpose of identifying animals based on genetic propensities. Other data or databases include, but are not limited to, those containing information related to DNA testing, vaccination, SUREBRED pre-conditioning program, estrus and pregnancy results in non-poultry animals, hormone levels, food safety/contamination, somatic cell counts, mastitis occurrence, diagnostic test results, milk protein levels, milk fat, vaccine status, health records, mineral levels, trace mineral levels, herd performance, and the like. For poultry, such databases may include, for example, husbandry conditions, food safety/contamination, vaccine status, health records, mineral levels, trace mineral levels, flock performance, and the like.

The present invention encompasses computer-assisted methods for tracking the breeding and veterinary histories of livestock animals comprising using an interactive computer system such as, for example, a programmed computer comprising a processor, a data storage system, an input device and an output device, comprising the steps of inputting into the programmed computer through the input device data comprising a breeding history of a livestock animal or population of livestock animals, inputting into the programmed computer through the input device data comprising a veterinary history of a livestock animal or population of livestock animals, correlating the veterinary data with the breeding history of the livestock animal or population of livestock animals using the processor and the data storage system and outputting to the output device the breeding history and the veterinary history of the livestock animal or population of livestock animals.

The databases and the analysis thereof will be accessible to those to whom access has been provided, preferably by means of a secure log-in procedure. Access can be provided through rights to access or by subscription to specific portions of the data. For example, the database can be accessed by owners of the animal, the test site, the entity providing the sample to the test site, feedlot personnel, and veterinarians. The data can be provided in any form such as by accessing a website, fax, email, mailed correspondence, automated telephone, or other methods for communication. Particularly advantageous is a PDA or pocket computer. Such a portable device may be stand-alone and the operator can only access and interact with data already stored in the device, either in solid-state memory, a hard-drive or other data storage means that does not encumber the portability of the computer device. It is also within the scope of the invention for the portable computing device to access the internet, preferably, but not only, via a wireless connection. This data can also be encoded on a portable storage device, such as a microchip or RFID tag, that can be implanted in the animal. Advantageously, information can be read and new information added without removing the microchip from the animal.

The present invention comprises systems for performing the methods disclosed herein. Such systems comprise devices, such as computers, internet connections, servers, and storage devices for data. The present invention also provides for a method of transmitting data comprising transmission of information from such methods herein discussed or steps thereof, e.g., via telecommunication, telephone, video conference, mass communication, e.g., presentation such as a computer presentation (e.g. POWERPOINT), internet, email, documentary communication such as a computer program (e.g. WORD) document and the like.

Systems of the present invention comprise a data collection module, which includes a data collector to collect data from an animal or embryo and transmit the data to a data analysis module, a network interface for receiving data from the data analysis module, and optionally further adapted to combine multiple data from one or more individual animals, and to transmit the data via a network to other sites, or to a storage device.

More particularly, systems of the present invention comprise a data collection module, a data analysis module, a network interface for receiving data from the data analysis module, and optionally further adapted to combine multiple data from one or more individual animals, and to transmit the data via a network to other sites, and/or a storage device. For example, the data collected by the data collection module leads to a determination of the absence or presence of a gene in the animal or embryo, and for example, such data can then be transmitted to a feedstock site when the feeding regimen of the animal is planned.

In one example, animals that are predisposed to a disease based upon their genetic profiles can be targeted for vaccine or antibiotic treatment as well as a visit by a veterinarian. Instead of treating every bird with antibiotics, the farmer can identify those individual birds predisposed to a disease using, for example, the methods as described, for example in U.S. patent application Ser. No. 60/560,115 incorporated herein by reference in its entirety. Thus, the farmer can minimize costs by sending only the animals genetically predisposed to a disease to a veterinarian instead of the entire flock. Furthermore, especially in an embodiment where the data is implanted on a microchip on a particular animal, the farmer can optimize the efficiency of managing the herd because the farmer is able to identify the genetic predispositions of an individual animal as well as past, present and future treatments (e.g., vaccinations and veterinarian visits).

The invention also provides for accessing other databases such as herd or flock data relating to genetic tests and data performed by others, by datalinks to other sites. Therefore, data from other databases can be transmitted to the central database of the present invention via a network interface for receiving data from the data analysis module of the other databases.

The invention further relates to a computer system and a computer readable media for compiling data on a bird or flock of birds, the system containing inputted data on that animal(s), such as vaccination and medication histories, DNA testing, food safety/contamination, somatic cell counts, diagnostic test results, egg yield, vaccine status, health records, mineral levels, trace mineral levels, herd performance, and the like. The data of the animal can also include prior treatments as well as suggested tailored treatment depending on the genetic predisposition of that animal toward a particular disease.

A computer readable media may contain such data as described above. The invention also relates to a method of doing business comprising providing to a user the computer system described herein or the media described herein.

The invention provides for a computer-assisted method for tracking the breeding and veterinary histories of poultry comprising using an interactive computer system, e.g., a programmed computer comprising a processor, a data storage system, an input device and an output device, comprising the steps of inputting into the programmed computer through the input device data comprising a breeding history of a bird or flock of birds, inputting into the programmed computer through the input device data comprising a veterinary history of a bird or flock of birds, correlating the veterinary data with the breeding history of the bird or flock of birds using the processor and the data storage system and outputting to the output device the breeding history and the veterinary history of the bird or flock of birds.

The invention also provides for a computer-assisted method for improving animal production comprising using an interactive computer system, e.g., a programmed computer comprising a processor, a data storage system, an input device and an output device, the steps of inputting into the programmed computer through the input device data comprising a breeding, veterinary, medication, diagnostic data and the like of an animal, correlating a physical characteristic predicted by the genotype using the processor and the data storage system, outputting to the output device the physical characteristic correlated to the genotype and feeding the animal a diet based upon the physical characteristic, thereby improving livestock production.

The invention further provides for a computer-assisted method for optimizing efficiency of feed lots for livestock comprising using a computer system, e.g., a programmed computer comprising a processor, a data storage system, an input device and an output device, and the steps of inputting into the programmed computer through the input device data comprising a breeding, veterinary etc history of an animal, correlating the breeding, veterinary etc histories using the processor and the data storage system, outputting to the output device the physical characteristic correlated to the genotype and feeding the animal a diet based upon the physical characteristic, thereby optimizing efficiency of feed lots for livestock.

It is contemplated that the business methods of the invention encompasses interactive systems whereby a series of questions may be posed to the operator or the operator may exchange data with a database specific to the flock of subject animals. By means of an interactive system, responses to the questions may be input into the method to provide specific optimization of the feed, treatment, etc of the subject animals. Such interactive systems may be wireless devices that would permit a farmer to readily obtain relevant information while in the field and even communicate and interact with a human operator able to provide further specific information and advice.

The invention further encompasses methods of doing business by providing access to such computer readable media and/or computer systems and/or data collected from animals to users; e.g., the media and/or sequence data can be accessible to a user, for instance on a subscription basis, via the Internet or a global communication/computer network; or, the computer system can be available to a user, on a subscription basis.

Another aspect of the invention encompasses an interactive system wherein the computer-based system of the invention may further encompass a means of inputting data into the system in response to a demand such as a question. The interactive system, therefore, may encompass an output in the form of questions relating to the ability of a student to understand or operate the computer system and the inputting of the response of the student to the question. The interactive system of the invention may encompass a means of receiving the input from multiple stations such multiple students, the means of inputting being, for example a wireless transmitter configured to allow a student to respond to a multiple-choice question presented by the instructor. The interactive system will allow the operator such as an instructor to modify the instruction of the computer-based system immediately according to the progress of the students.

The input of the responses from the students may be presented, for example, as a numeric value or graphically, such as a in the form of a pie-chart. The interactive system need not present the accumulated response data to the respondents. In this case the interactive system of the invention may be useful in providing training of the instructors without modifying the interaction of the students themselves. The interactive system according to the invention may also be useful to determine the response of the respondents to a new product such as a veterinary product, whereby the data output relevant to a flock of birds from the computer-based methods of the invention is presented to the operators and then the effect of the new product is introduced. The interactive system may allow the presenter to assess the acceptance or otherwise of the operators to the new product and its effect on the flock.

In one embodiment, the invention provides for a computer system for managing livestock comprising physical characteristics and databases corresponding to one or more animals. In another embodiment, the invention provides for computer readable media for managing livestock comprising physical characteristics and veterinary histories corresponding to one or more animals. The invention further provides methods of doing business for managing livestock comprising providing to a user the computer system and media described above or physical characteristics and veterinary histories corresponding to one or more animals.

Accordingly, the invention further encompasses methods of transmitting information obtained in any method or step thereof described herein or any information described herein, such as via telecommunications, telephone, mass communications, mass media, presentations, internet, email and the like.

One aspect of the present invention, therefore, is computer-assisted methods for the tracking and correlating of breeding, veterinary care and performance data relating to one or more livestock animals. In particular, the methods are especially useful for tracking the breeding and veterinary care histories of individual animals or the collective flock and relating the histories to the performance parameters of the animals and to the expected or desired performances. The methods of the invention can allow the operator to be alerted by deviation of the actual performance parameters from the desired performances so that the feed, medications, vaccinations etc of the animals may be adjusted or corrected accordingly.

One embodiment of the invention is a computer-assisted method for tracking the breeding and veterinary histories of livestock animals comprising using a computer system, e.g., a programmed computer comprising a processor, a data storage system, an input device and an output device, comprising the steps of inputting into the programmed computer through the input device data comprising a breeding history of a bird or flock of birds, inputting into the programmed computer through the input device data comprising a veterinary history of a bird or flock of birds, correlating the veterinary data with the breeding history of the bird or flock of birds using the processor and the data storage system and outputting to the output device the breeding history and the veterinary history of the bird or flock of birds.

In one embodiment of the invention, the method further comprises the steps of inputting into the programmed computer diagnostic data related to the health of the livestock animal or population of livestock animals; and correlating the diagnostic data to the breeding and veterinary histories of the livestock animal or population of livestock animals.

In the various embodiments of the invention, the veterinary data can include, but is not limited to, a vaccination record for a bird or flock of birds. The health data may comprise other than the vaccination record for a bird or flock of birds such as, but not limited to, records of medications administered to the animal(s), infections or other injuries and surgical procedures.

In other embodiments of the methods according to the invention, steps may be included of inputting into the programmed computer data related to the welfare of the bird or flock of birds; and correlating the welfare data to the breeding and veterinary histories of the bird or flock of birds.

In still other embodiments of the invention, the methods may further comprise the steps of inputting into the programmed computer data related to the quality control of the bird or flock of birds; and correlating the quality control data to the breeding and veterinary histories of the bird or flock of birds.

The method according to the invention may also comprise the steps of inputting into the programmed computer performance parameters of the livestock animal or population of livestock animals; and correlating the required performance parameters of the livestock animal or population of livestock animals to a specific desired performance requirement of a customer. The embodiments may optionally further comprise correlating the vaccine data to the performance parameters of the livestock animal or population of livestock animals and/or inputting into the programmed computer data related to the nutritional data of the livestock animal or population of livestock animals; and correlating the nutritional data to the performance parameters of the livestock animal or population of livestock animals.

Yet other embodiments of method according to the invention further comprise the step of alerting the system operator to undesirable changes in the performance parameters of the livestock animal or population of livestock animals that could allow the operator to modify the feed and medication of the animals accordingly. The embodiments of the invention may further comprise the steps of inputting into the programmed computer through the input device data comprising a genotype of an animal, correlating a physical characteristic predicted by the genotype using the processor and the data storage system, and outputting to the output device the physical characteristic correlated to the genotype for a livestock animal or population of livestock animals and feeding the animal(s) a diet based upon the physical characteristic, thereby improving livestock production.

Still other embodiments of the computer-assisted methods according to the invention optimize the efficiency of feed lots for livestock and comprise the steps of outputting to an output device the breeding and veterinary history of the livestock animal or population of livestock animals, and feeding the animal(s) a diet based upon their breeding and veterinary histories, thereby optimizing efficiency of feed lots for the livestock animal or population of livestock animals.

It is intended that the computer-assisted methods of the present invention are suitable for collating, analyzing and providing data on any livestock animal including, but not limited to a bird or flock of birds such as chicken, turkey, duck and the like.

Another aspect of the present invention is a method of transmitting data comprising transmission of information from such methods herein discussed or steps thereof, e.g., via telecommunication, telephone, video conference, mass communication, e.g., presentation such as a computer presentation (for example, a POWERPOINT™ presentation), internet, email, documentary communication such as a computer program document and the like.

Another aspect of the invention is a computer system for tracking breeding and welfare histories of livestock comprising breeding and veterinarian data corresponding to one or more livestock animals. Yet another aspect of the invention is computer readable media for tracking breeding and welfare histories of livestock comprising breeding and veterinarian data corresponding to one or more livestock animals. Still another aspect of the invention is methods of doing business for tracking breeding and welfare histories of livestock comprising breeding and veterinarian data corresponding to one or more livestock animals comprising providing to a user the computer system of claim 14 or the media of claim 15.

In one aspect of the invention, the business method further comprises an interactive system that permits a comparison of the evolution and reception of the message desired to be presented to a body of students undergoing training in the use of the business methods of the invention. The interactive system may also be useful in the field, whereby an operator of the computer-assisted business method of the present invention may respond to questions intended to assist in the operation of the method or to optimize the method to correspond to the particular needs of the operator. It is contemplated, for example, that the students may receive a first presentation, be questioned as to the extent of their learning, the responses to the questions be inputted via an interactive device such as, but not limited to, a POWERVOTE™ device (La Generale Multimedia, Clichy cedex, France).

Any input device and software may be used that allows a user to respond to a question with, for example, a yes, no or do not know response, or to select from a list of possible answers. The device or devices may be connected to the computer system of the present invention or to a computer providing the presentation by cable or by wireless means. The interactive system of the present invention is useful to provide interactive presentations for students of the business method of the invention or for customers, whereby the computer-assisted methods can be modified according to the responsive input from the interactive systems to better suit the needs of the student or the customer. The interactive system of the invention further provides for the output of data from the interactive system in the form of notepad files or other graphical means of data presentation such as pie or bar charts. The input of data from the interactive system may also allow for comparison of the input results received during a presentation to be compared with national or global data allowing local, regional and international use of the business methods of the invention to be compared. It is contemplated that any language may be used with the methods and interactive system of the invention

The interactive system of the invention is especially advantageous for the presentation to prospective customers of a new product that may be used for the improvement of the health or productive yield of a flock of birds. For example, during the internal training of sale representatives, a first set of questions concerning the operation of the computer-assisted methods or the data output therefrom can be responded to by the trainees without displaying the results. A POWERPOINT™ presentation for the launch of a new product is then shown and the trainees are then asked the same questions with the display of the results of both question times, thereby demonstrating the advantages or expected effects of the new product on the output data from the computer-assisted methods. The system may also be configured to direct the system operator, such as a veterinarian, to a specific medication or mode of treatment more appropriate for the treatment of the subject animals.

Another aspect of the invention is a method for entering and retrieving data relevant to a flock of birds into a computer device such as, but not limited to, a personal digital assistant (PDA) or a pocket computer that may be readily transported to the location of the flock to provide instant access by the operator to information relevant to the maintenance and rearing of the birds. The method may also provide a firewall protected access to the internet to further expand the available information and to allow suppliers of, for example, veterinarian products to suggest suitable products and dosages useful for administering to the birds. The computer device may also be configured to synchronize with a central server to deliver data to the device and to permit the uploading of data to the server for storage and/or subsequent analysis. The methods of the invention as incorporated into a readily portable computer device are especially useful to a veterinarian making field visits to the site of the bird rearing house or shed. The software of the method may be configured to allow veterinarians to set their personal data and preferences, including name, address, telephone number and the like. Data concerning the company raising the birds and which may also be entered into the device may be, but are not limited to, creating a new company entry, updating and/or consulting company information, consulting the company policies as to the protocols and outcome requirements for the rearing of the animals, stocking density, live weight, feed policy, average feed consumption, coccidiostat policy, feed code, mortality rate, hatched gender ratio and inclusion rate.

The veterinarian or other operator of the method may also create a new farm organization entry to update or consult farm organization information, including the links to companies operating the farms. A given farm organization could be linked to one or more companies.

A user management field of data entry allows the avian application administrator to define and authorize veterinarian users to access the application, preferably by means of a login password.

A prescription editor will allow the avian application administrator to define the required prescription print-out (fields, fonts, etc). Changes made in this field may be communicated back to the computer device users during their synchronization. A data analysis option will allow the avian application administrator to access reports based on data entered by the veterinarian users in the fields.

Once logged in the system, the farm selection option is the entry point for the core of the application. It allows the veterinarian to select an existing farm within his (her) customer base or create a new farm entry. Selection on existing farms may be based on farm name and/or town. Once a farm is selected or created, the veterinarian may go to the Prescription sub menu or to the Information Management sub-menu associated with the particular farm.

One sub-menu (farm-farm information) presented to the operator, allows the veterinarian to define and/or update the farm structure from the owning company to the house or shed, all relevant information being captured through this menu's options as follows: farm information (create and/or update) option allows to manage farm information as well as the web-based links to the owning company and farm organization(s); house and/or shed information (create and/or update) would allow management of the house and/or shed information linked to the selected or created farm; feed bin information (create or/and update option would allow management of the Feed Bin information linked to the selected or created house and/or shed; bird batch information (create or/and update) option allows management of bird batch information linked to the selected house and/or shed. The information managed through this option can evolve over time but historical values will only be kept in the accessible database for a bird batch level, e.g. if for a given house, a new vaccination or medication tank is installed, only the new value for this data will be used. All values associated to bird batches will be retained for future analysis.

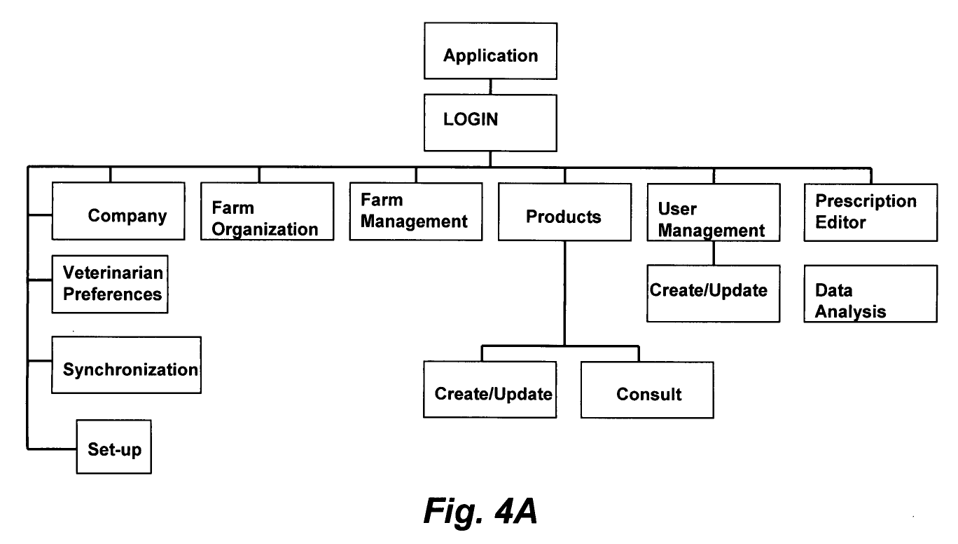

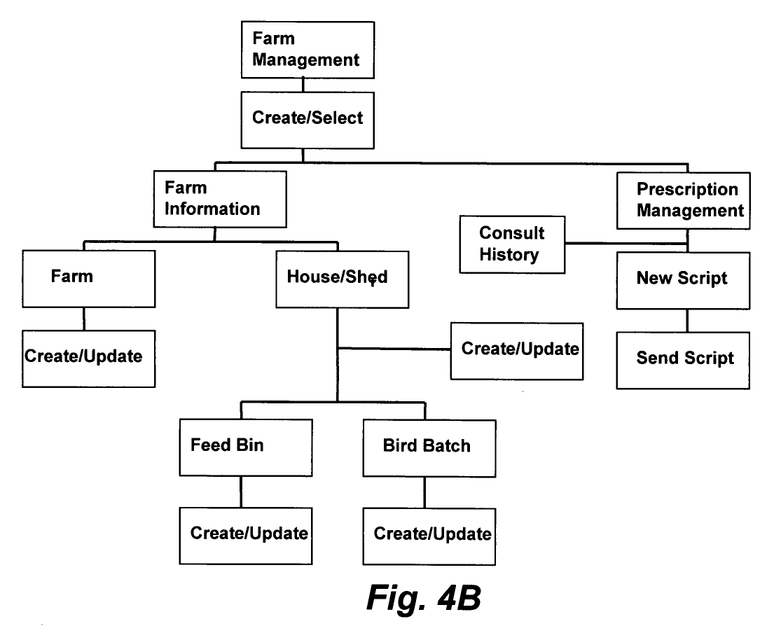

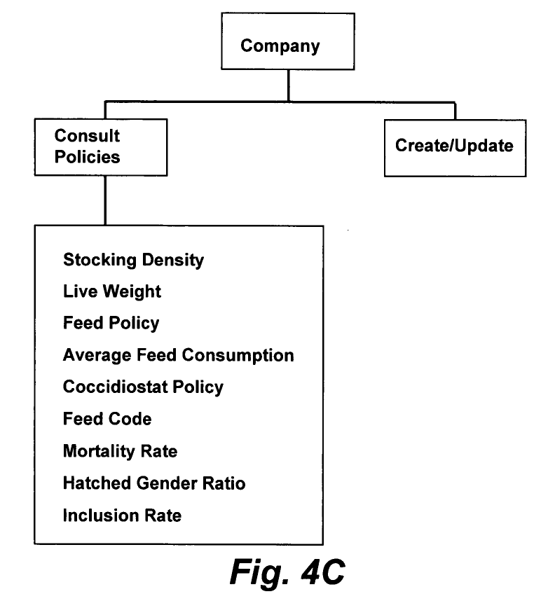

A ‘Farm-Prescription Management’ sub-menu can allow the veterinarian to consult a previous prescription for a given farm/house or bird batch or write and send a new prescription at the farm level or for one or several houses, i.e. bird batch(es). In addition, a Farm-Prescription Management-Consult History option will allow the veterinarian or other operator to select and consult a past prescription. A list of past prescriptions for the selected farm may be displayed with their date and house number displayed (if several houses, numbers are be comma separated). The list optionally can be sorted by date descending order. By clicking on one element of the list, the operator will see the prescription details. A Farm-Prescription Management-New script option will allow the operator to write and send a new prescription. A flow chart of options available in the system of the invention is presented in FIGS. 4A-4C.

In addition, a ‘Prescription Editor’ option will allow a system administrator to adapt the format and layout of the various prescriptions. For example, but not intended to be limiting, options for this component of the application include an export function wherein the user may select and export all the information required to an EXCEL™ format to output the data as needed (graphical format, report format, etc), or in an analysis function, whereby the operator may select data and reports to be built on the web using selected results.

A ‘Data Analysis’ option will allow a system administrator to consult and analyze the data entered by multiple operators of the computer devices such as veterinarians using the Pocket PC application of the invention. Options for this component of the application also may include an export function wherein the user may select and export all the information required to an EXCEL™ format to output the data as needed (graphical format, report format, etc), or in an analysis function, whereby the operator may select data and reports to be built on the web using selected results.

The term ‘data elements’ as used herein refers to a commercial company, a farm, breeding or rearing station and the like. A farm, for example, may have one or more attributes such as, but not limited to, a farm name, an address, the attributes being linked by relationships. For example, a shed used for the rearing or maintenance of a flock of birds may belong to only one farm).

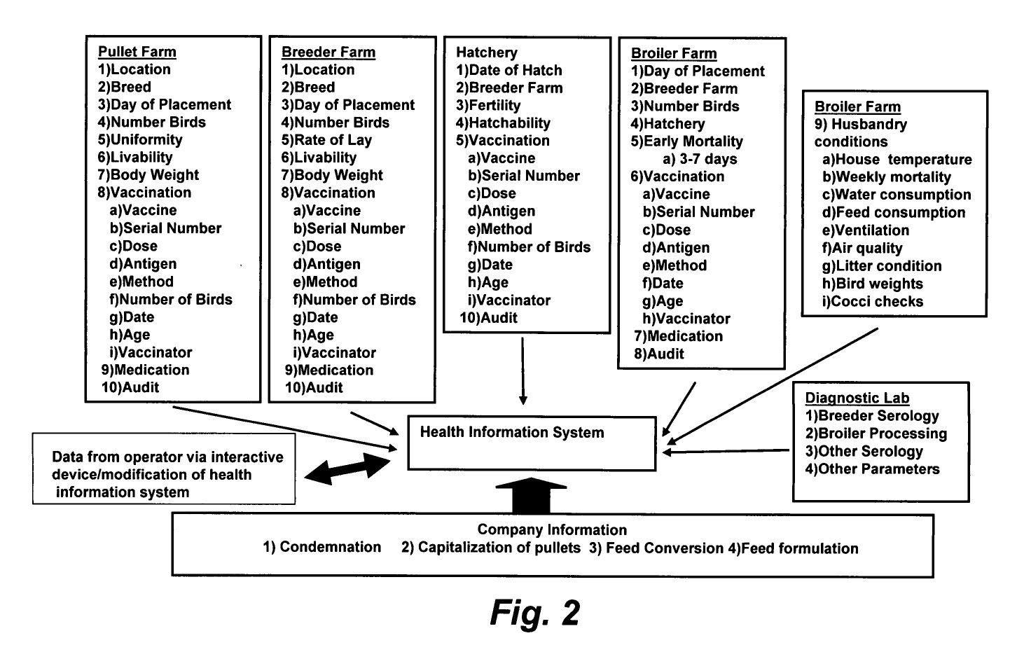

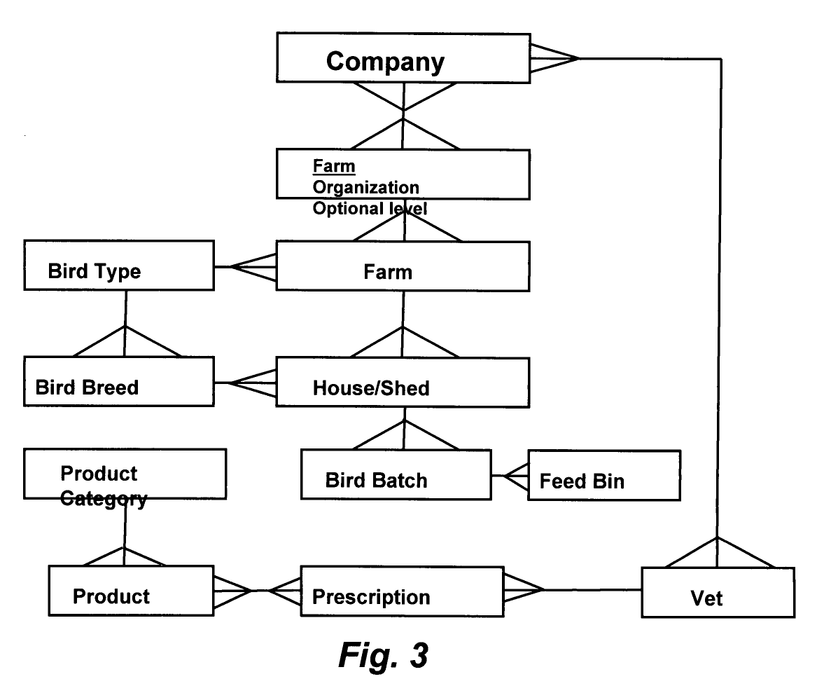

Information describing a company that may be entered into the computer-based system includes, but is not limited to, or necessarily includes the following: name of company, address 1, address 2, town, post code, county, country, map reference, e-mail address, telephone number, fax number, mobile phone number. Similar data may be entered as it relates to veterinarian(s) attending to the care of the flock. An optional data entry may relate to the farm organization, wherein one farm may provide birds for delivery to at least two companies. Potential relationships between the data elements are illustrated in FIGS. 2 and 3.

The methods of the present invention also provide for the entry of comments related to one or more of the data entries relating to a company or farm. For example, a data field associated with a farm address may be the region of the country and include a comment regarding the Coccidiostat policy of the region. Another example of a data entry line may be for the particular bird type (species, breed, etc) and the comment line could indicate whether the farm houses just this type or others. It is further contemplated that over time, the data will evolve. For example, a farm may initially only work for one company at a time. However, over time, a Farm may work for several companies. Alternatively, a company may have several farms.

Each company will have specific policies, protocols and/or standards for the rearing of birds that may evolve over time, as shown in Example 2, Table 1 below. Other exemplary data entries may include, but are not limited to, data concerning houses or sheds used for animal maintenance, bird batch, feed bins, bird type, bird breed, or a veterinary product category or product administered to the birds and shown in Example 3 below. Product categories include, but are not limited to, in-feed therapeutics, in-feed coccidiostats, in-feed digestive enhancers, in-feed antihelmintics, water-soluble antibiotics, water-soluble coccidiostats, water-soluble vitamins and minerals, water-soluble antihelmintics, live vaccines, inactivated vaccines.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

### EXAMPLE 1

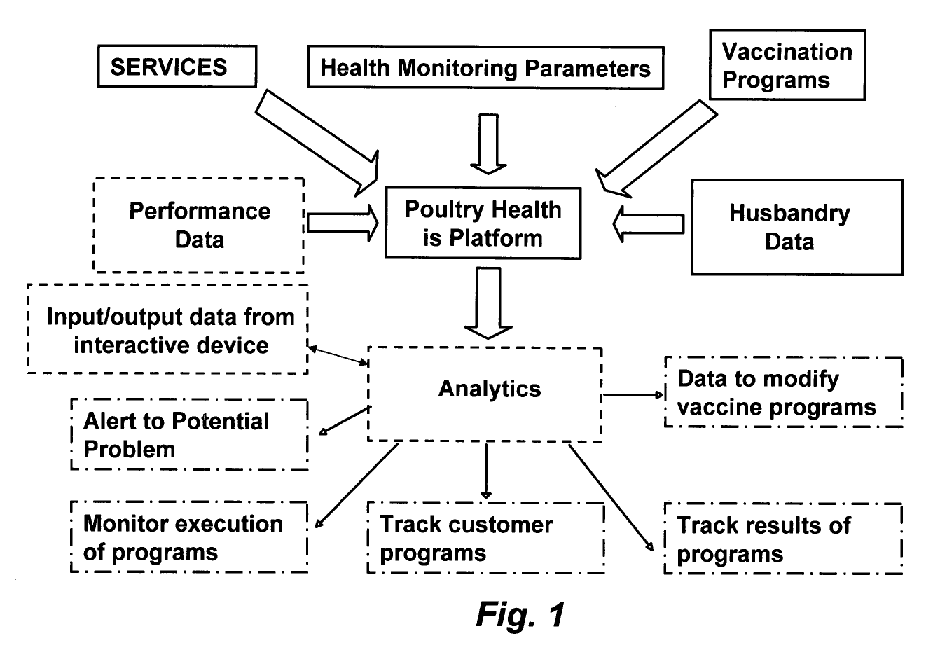

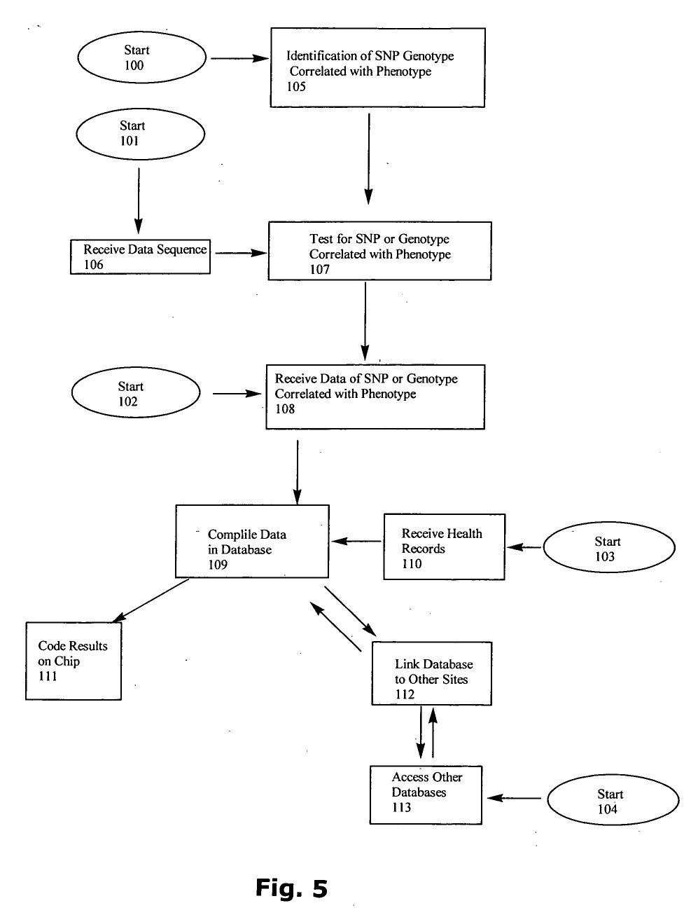

FIG. 1 shows a flowchart of the input of data and the output of results from the analysis and correlation of the data pertaining to the breeding, veterinarian histories and performance requirements of a group of animals such as from a flock of birds. FIG. 2 illustrates a flowchart for the input of data detailing the breeding history and the vaccination records of a flock of birds. The flowcharts shown in FIGS. 1 and 2 may further comprise the input of genotype data as described in U.S. patent application Ser. No. 60/560,115 incorporated herein by reference in its entirety. The flowcharts illustrated in FIGS. 1 and 2 further indicate the interactive flow of data from the computer-assisted device to a body of students learning the use of the method of the invention and the correlation of such interactive data to present an output as a pie-chart indicating the progress of the class. The flowchart further indicates modifications of the method of the invention in accordance with the information received from the students to advance the teaching process or optimize the method to satisfy the needs of the students. FIG. 5 illustrates a flow chart of the input of data and the output of results from the analysis and the correlation of the data pertaining to the breeding, veterinarian histories, and performance requirements of a group of animals.

### EXAMPLE 2

### An Application of Policies, Protocols or Standards as Applied to the Rearing of a Population of Birds by a Company

Feed policy may be given for each bird breed and age, therefore, the relationships will be expressed as a matrix with bird breed and age being the axis.

TABLE 1

| Policy/ | Bird | Bird | | | | |
|---|---|---|---|---|---|---|
| Standard | Type | Breed | Age | Region | Product | Comment |
| Stocking density | XX | X | | X | | |
| Live weight | | XX | XX | | | |
| Feed policy | | XX | XX | X | | |
| Average Feed | | XX | XX | X | | |
| Consumption | | | | | | |
| Water | | | | | | |
| Consumption | | | | | | |
| Coccidiostat | | XX | XX | XX | | |
| policy | | | | | | |
| Feed code | | XX | XX | XX | | |
| Mortality Rate | | XX | XX | | | |
| Hatched gender | | XX | | | | |
| ratio | | | | | | |
| Inclusion Rate | XX | | XX | | XX | |

XX: Mandatory data entry
X: Optional

### EXAMPLE 3

### Exemplary Data Entries Relevant to a Specific Company or Farm

TABLE 2
House/Sheds

| Field | Comment |
|---|---|
| N^{o} | |
| Name | |
| Floor Area | In m² |
| N^{o} of Birds | Floor Area × Stocking Density |
| | for the Bird Type by Company |
| | policy |
| Feeder Type (Track or | Track or Pan |
| Pan) | |
| Main Water Source | Mains/Bore Hole/Other |
| Header Tank | Yes or No |
| Size Header Tank | Litre or Gallon |
| Header Tank Location | Shed/Control Room/Outside |
| Drinker System Type | Nipple with drip tray/Nipple |
| | without drip tray |
| Vaccination/Medication | Yes or No |
| Tank | |
| Continuous Dosing | Yes or No |
| machine | |

A house/shed typically belongs to only one farm. A house/shed typically may produce birds for only one company. A house/shed produce bird batches and a farm can have several houses/sheds.

TABLE 3
Bird Batch

| Field | Comment |
|---|---|
| N^{o} | |
| Starting Week # | |
| Ending Week # | |
| Bird Breed | |

A bird batch is made within one house/shed. A bird batch typically contains only one bird breed.

TABLE 4
Feed bins

| Field | Comment |
|---|---|
| No | |
| Capacity | |

A house/shed may have several feed bins. A feed bin may belong to only one house/shed.

TABLE 5
Bird Type

| Field | Comment |
|---|---|
| Name | |

A bird type may have several bird breeds. A given farm at a given time may only produces one bird type within the houses/sheds. Possible bird types include, but are not limited to: broiler breeder, turkey breeder, turkey grower, broiler, layer, pullet, pullet layer, pullet breeder, duck breeder, Peking duck breeder, Muscovy duck breeder, Muscovy duck grower, pheasant, partridge, guinea fowl breeder, guinea fowl grower. A bird breed belongs to only one bird type.

TABLE 6
Product

| Field | Comment |
|---|---|
| Brand Name | |
| Active Name/Ingredient | |
| Concentration of Active | |
| ingredient | |
| Product Type | in-feed, water-soluble or |
| | vaccine |
| Species | |
| Dose rate of active | |
| Dosing instructions | Must include options for |
| | pulse/continuous for water |
| | soluble products |
| Pack size available | |
| Marketing | |
| Authorization number | |
| Withdrawal period | Meat Withdrawal period |
| Meat | |
| Withdrawal period Egg | Eggs Withdrawal period |
| Cost per unit | |

While the invention has been described with reference to specific methods and embodiments, such description is for illustrative purposes only. The words used are words of description rather than of limitation. It is to be understood that changes and variations may be made by those of ordinary skill in the art without departing from the spirit or scope of the present invention, which is set forth in the following claims.

What is claimed is:

1. An interactive computer-assisted method for tracking the rearing of livestock birds comprising, using a computer system comprising a programmed computer comprising a processor, a data storage system, an input device, an output device, and an interactive device, the steps of: (a) inputting into the programmed computer through the input device data comprising a breeding history of a bird or flock of birds, (b) inputting into the programmed computer through the input device data comprising a veterinary history of a bird or flock of birds, (c) correlating the veterinary data with the breeding history of the bird or flock of birds using the processor and the data storage system, and (d) outputting to the output device the breeding history and the veterinary history of the bird or flock of birds.

2. The method according to claim 1, wherein the computer system is an interactive system whereby modifications to the output of the computer-assisted method may be correlated according to the input from the interactive device.

3. The method according to claim 1, further comprising the steps of inputting into the programmed computer diagnostic data related to the health of the bird or flock of birds; and correlating the diagnostic data to the breeding and veterinary histories of the bird or flock of birds.

4. The method according to claim 1, wherein the veterinary data comprises a vaccination record for a bird or flock of birds.

5. The method according to claim 3 wherein the health data is selected from the group consisting of husbandry condition data, flock history, and food safety data.

6. The method according to claim 1, further comprising at least one further step selected from the group consisting of inputting into the programmed computer data related to the quality control of the bird or flock of birds and correlating the quality control data to the breeding and veterinary histories of the bird or flock of birds, inputting into the programmed computer performance parameters of the bird or flock of birds; and correlating the required performance parameters of the bird or flock of birds to a specific performance requirement of a customer, correlating the vaccine data to the performance parameters of the bird or flock of birds, correlating flock to the performance parameters of the bird or flock of birds, correlating the food safety data to the performance parameters of the bird or flock of birds, correlating the husbandry condition data to the performance parameters of the bird or flock of birds, inputting into the programmed computer data related to the nutritional data of the bird or flock of birds; and correlating the nutritional data to the performance parameters of the bird or flock of birds, and alerting to undesirable changes in the performance parameters of the bird or flock of birds.

7. The method according to claim 1, further comprising the steps of inputting into the programmed computer through the input device data comprising a genotype of a bird; correlating a physical characteristic predicted by the genotype using the processor and the data storage system; and outputting to the output device the physical characteristic correlated to the genotype for a bird or population of birds, and feeding the animal(s) a diet based upon the physical characteristic, thereby improving avian production.

8. The computer-assisted method according to claim 1 for optimizing efficiency of feed lots for livestock comprising outputting to the output device the breeding and veterinary history of the bird or flock of birds and feeding the animal(s) a diet based upon their breeding and veterinary histories, thereby optimizing efficiency of feed lots for the bird or flock of birds.

9. A method of transmitting data comprising transmission of information from such methods according to claim 1, selected from the group consisting of telecommunication, telephone, video conference, mass communication, a presentation, a computer presentation, a POWERPOINT™ presentation, internet, email, and documentary communication.

10. An interactive computer system according to claim 2 for tracking breeding and welfare histories of poultry comprising breeding and veterinarian data corresponding to a bird or flock of birds, and wherein the computer system is configured to allow the operator thereof to exchange data with the device or a remote database.

11. The interactive computer system according to claim 10, wherein the input and output devices are a personal digital assistant or a pocket computer.

12. A method of doing business for tracking breeding and welfare histories of livestock comprising breeding and veterinarian data corresponding to one or more livestock animals comprising providing to a user the computer system of claim 10.

13. A method of doing business for tracking breeding and welfare histories of livestock comprising breeding and veterinarian data corresponding to one or more livestock animals comprising providing to a user the computer system of claim 11.

14. The method of doing business according to claim 12, further comprising providing the animal owner or customer with sample collection equipment, such as swabs and vials useful for collecting samples from which genetic data may be obtained, and wherein the vials are optionally packaged in a container which is encoded with identifying indicia.

15. The method of doing business according to claim 1, wherein the computer system further comprises a plurality of interactive devices and wherein the method further comprises the steps of a receiving data from the interactive devices, compiling the data, outputting the data to indicate the response of a student or class of students to a question relating to the operation of the computer-assisted method, and optionally modifying the operation of the computer-assisted method in accordance with the indication of the response.
